(19) United States
(12) Patent Application Publication
(10) Pub. No.: US 2016/0148121 A1
(43) Pub. Date: May 26, 2016

(54) EMERGENCY-ROOM RESERVATION SYSTEM AND METHOD

(71) Applicants: Chris DURHAM, Atlanta, GA (US); Sahil PATEL, Atlanta, GA (US)

(72) Inventors: Chris DURHAM, Atlanta, GA (US); Sahil PATEL, Atlanta, GA (US)

(73) Assignee: ER EXPRESS, LLC, Atlanta, GA (US)

(21) Appl. No.: 14/554,771

(22) Filed: Nov. 26, 2014

(51) Int. Cl. G06Q 10/02; G06Q 50/22

(52) U.S. Cl. CPC G06Q 10/02; G06Q 50/22

(57) ABSTRACT

Reservations for emergency/urgent care are made by users using a queue feature that identifies earliest available reservations at nearby care facilities that are identified based on user-selected facility search parameters. The queue feature identifies the earliest available reservations based on user-selected treatment categories and facility-selected capacity characteristics for the treatment categories, and recommends a best reservation. The treatment categories can be for example adult, child/pediatric, urgent care, direct admit, walk-in, or worker's comp. And the capacity characteristics can be for example wait time, black-out period, and/or maximum number of reservations per time period. In addition, a reservation-update feature enables the care facilities to update the capacity characteristics for the treatment categories at their facilities to reflect changed conditions. And a reservation-confirmation feature enables the users to rerun the queue process to identify a new best reservation in light of the updated capacity characteristics for the treatment categories.

### CROSS-REFERENCE TO RELATED APPLICATIONS

This application claims the priority benefit of U.S. Provisional Patent Application Ser. No. 61/968,426, filed Mar. 21, 2014, and U.S. Provisional Patent Application Ser. No. 61/909,531, filed Nov. 27, 2013, both of which are hereby incorporated herein by reference.

### TECHNICAL FIELD

The present invention relates generally to systems and methods for reserving projected treatment times for healthcare, and particularly to computer-implemented systems and methods for online booking and managing of reservations for emergency/urgent care.

### BACKGROUND

Over the last decade, the number of visits to emergency departments (e.g., emergency rooms and urgent-care clinics) has increased by 32%, and the current number is expected to double over the next ten years. Concurrently, the number of emergency departments has decreased by about 4.6%. Consequently, by the year 2013 the average patient wait time for emergency care was nearly four hours, as reported in BECKER'S HOSPITAL REVIEW.

Accordingly, it can be seen that needs exist for solutions that reduce emergency-care wait times. It is to the provision of solutions to this and other problems that the present invention is primarily directed.

### SUMMARY

Generally described, the present invention relates to an online-reservation service that allows users to identify and book a best reservation option for emergency/urgent care, and to reschedule to a new best reservation if conditions change (for better or worse) at the selected or another nearby care facility after the reservation is booked. Reservations for emergency/urgent care are made by users using a queue feature that identifies earliest available reservations at nearby care facilities that are identified based on user-selected facility search parameters. The queue feature identifies the earliest available reservations based on user-selected treatment categories and facility-selected capacity characteristics for the treatment categories, and recommends a best reservation. The treatment categories can be for example adult, child/pediatric, urgent care, direct admit, walk-in, or worker's comp. And the capacity characteristics can be for example wait time, black-out period, and/or maximum number of reservations per time period. In addition, a reservation-update feature enables the care facilities to update the capacity characteristics for the treatment categories at their facilities to reflect changed conditions. And a reservation-confirmation feature enables the users to rerun the queue process to identify a new best reservation in light of the updated capacity characteristics for the treatment categories.

The specific techniques and structures employed to improve over the drawbacks of the prior art and accomplish the advantages described herein will become apparent from the following detailed description of example embodiments and the appended drawings and claims.

### BRIEF DESCRIPTION OF THE DRAWINGS

FIG. 1 is a schematic diagram of a user's portable electronic device enabled to implement an ER-reservation process according to an example embodiment of the present invention.

FIG. 2 is a front side view of the user device of FIG. 1.

FIG. 3 is a schematic diagram of system that includes the user device and an ER's electronic device to implement the ER-reservation process.

FIG. 4 is a flow diagram of the ER-reservation process implemented by the system of FIG. 3.

FIG. 5 is a flow diagram of a queue process of the ER-reservation process of FIG. 4.

FIG. 6 is a flow diagram of a reservation-confirmation process implemented by the system of FIG. 3 in conjunction with the ER-reservation process of FIG. 4.

FIG. 7 is a flow diagram of a reservation-update process implemented by the system of FIG. 3 in conjunction with the reservation-confirmation process of FIG. 6.

FIG. 8 is an example user-device screenshot displaying a hospital webpage with the ER-reservation application of FIGS. 4-7 embedded on the hospital website.

FIG. 9 is an example user-device “reservation options” screenshot (for a smart-phone web browser) displaying the hospital and reservation-time options recommended by the queue process of FIG. 5 of the ER-reservation process of FIG. 4.

FIG. 10 is an alternative example user-device “reservation options” screenshot (for a desktop web browser) displaying the hospital and reservation-time options recommended by the queue process of FIG. 5 of the ER-reservation process of FIG. 4.

FIG. 11 is an example user-device “reservation details” screenshot displaying additional information about the selected hospital and displaying a patient data form of the ER-reservation process of FIG. 4.

FIG. 12 is an example hospital-device “reservations” screenshot of the reservation-update process of FIG. 7.

FIG. 13 shows a portion of the example hospital-device “reservations” screenshot of FIG. 12.

FIG. 14 is an example hospital-device “wait-time control” screenshot displayed upon activating the “update wait time” button of FIGS. 12-13.

FIG. 15 is an example hospital-device “throttle control” screenshot displayed upon activating the “update throttle” button of FIG. 12.

FIG. 16 shows a blackout-control portion of the example hospital-device “reservations” screenshot of FIG. 12

FIG. 17 is an example hospital-device “blackout control” screenshot displayed upon activating the “edit entire schedule” button of FIGS. 12 and 16.

FIG. 18 is an example referrer-device “referral” screenshot of a referring physician module that can be included in the ER EXPRESS of FIGS. 1-17.

### DESCRIPTION OF EXAMPLE EMBODIMENTS

The present invention relates to an online-reservation system and method that allows users (e.g., patients or caretakers) to identify and compare nearby hospitals in order to bypass a backlogged hospital ER waiting room, thereby providing patients with an alternative to the crowded ER waiting room. As used herein, the terms emergency room, ER, emergency department, ED, hospital, urgent-care facility, urgent-care clinic, and the like are sometimes used interchangeably. These terms are all intended to refer to a facility that provides emergency healthcare services, and the terms “hospital” and “emergency room,” and the designation “ER,” as used herein are intended to be broadly construed to cover all of these, unless clearly indicated otherwise in a specific instance. In addition, each of the various aspects of the invention described herein can be considered as inventions by themselves or in combinations.

The online-reservation system and method are implemented by a main reservation software program that resides on a server computer (e.g., a bank of computers in a centralized or distributed network) and that includes application modules that are downloaded and stored on respective user and hospital electronic devices, with the user and hospital applications communicating with the main reservation program for example using a wireless connection (e.g., Internet or cellular) or a wired connection. In other embodiments, the main reservation program is a mobile-optimized web application, with the user and hospital application modules not downloaded but instead residing on the server and remotely accessed by the user and hospital devices via a wireless or wired connection. It should be noted that the beneficial services provided to users and hospitals who use the online-reservation system, method, and software program are sometimes referred to herein generally as the ER EXPRESS service.

The electronic user devices are typically provided by mobile devices such as smart-phones, tablet computers, and/or laptop computers, typically with a GPS or other location-sensing device. In some embodiments, the user devices are provided by desktop computers or other non-mobile devices, with or without location-sensing capability, as noted below.

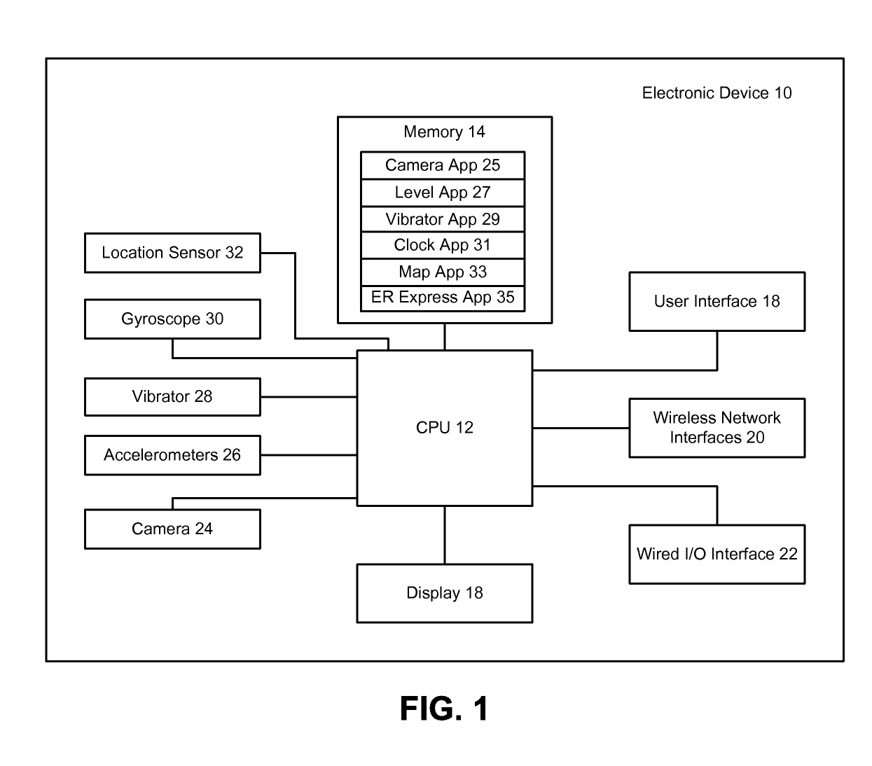

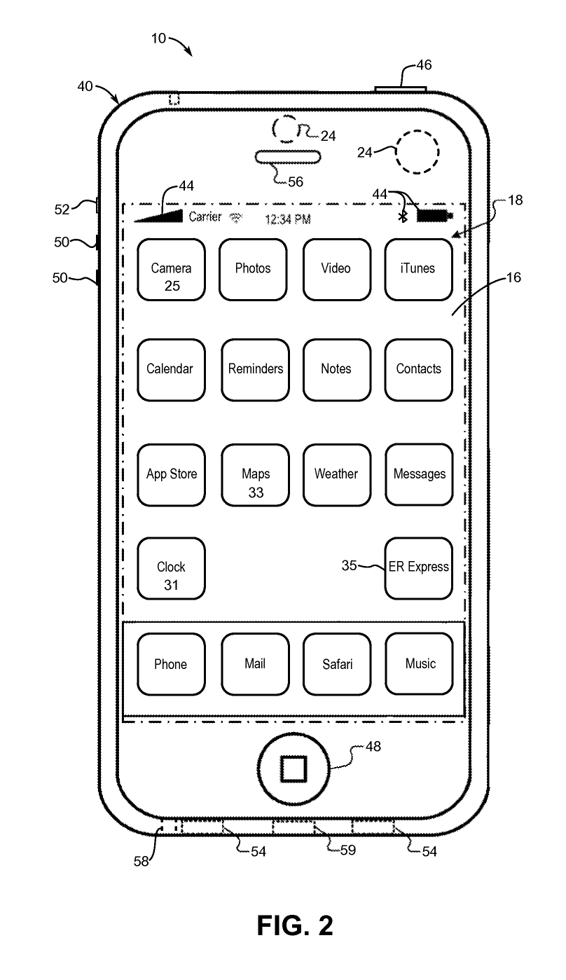

Referring now to the drawings, FIGS. 1-2 show a portable electronic device 10 adapted to provide certain ER-reservation functionality according to an example embodiment of the present invention. Referring particularly to FIG. 1, in the depicted embodiment, the electronic device 10 is a cellular phone (e.g., an IPHONE device). In other embodiments, the electronic device is a personal email device (e.g., a BLACKBERRY device), a personal media player (e.g., an IPOD device), a tablet computer (e.g., an IPAD device), a laptop computer, a personal digital assistant, a digital camera, or another electronic device.

The electronic device 10 of the depicted embodiment includes at least one central processing unit (CPU) 12, non-transitory memory storage device 14, display 16, user interface 18, wired input/output (I/O) interface 20, network interface 22, camera 24, accelerometer 26, vibrator 28, gyroscope 30, and location sensor 32. These components can be of a conventional and commercially-available type, as is known to persons of ordinary skill in the art. The CPU 12 includes one or more microprocessors, and the microprocessors may be “general purpose” microprocessors, a combination of general- and special-purpose microprocessors, or application-specific integrated circuits (ASICs). Additionally or alternatively, the CPU 12 may include one or more reduced instruction set (RISC) processors, video processors, or related chip sets. The CPU 12 provides processing capability to execute an operating system, run various applications, and/or provide processing for one or more of the processes described herein. Example applications that may run on the electronic device 10 typically include a music player, a video player, a picture displayer, a calendar, an address book, an email client, a telephone dialer, and so forth.

The memory storage device 14 is communicably coupled to the CPU 12 and stores data and executable code. The memory 14 typically includes volatile memory, such as random-access memory (RAM), and nonvolatile memory, such as read-only memory (ROM) or flash memory. In buffering or caching data related to operations of the CPU 12, the memory 14 may store data associated with open applications running on the electronic device 10. Being well-suited to long-term storage, the nonvolatile memory may store software (e.g., for implementing functions on the electronic device 10), data files (e.g., media such as music and video), preference information (e.g., ringtones and media playback preferences), transaction information (e.g., credit-card data and records of transactions), wireless connection information (e.g., wireless network names and passwords, and cellular network connections), subscription information (e.g., a record of subscribed-to podcasts or television shows), and personal information (e.g., contacts, calendars, and email).

The display 16 displays images and/or data to a user. The display 16 may be any suitable display, such as a liquid-crystal display (LCD), a plasma display, or a light-emitting diode (LED) display. In some embodiments, the display 16 includes touch-screen technology through which a user can interface with the electronic device 10.

The user interface 18 may include, for example, indicator lights, user inputs, and/or a graphical user interface (GUI) on the display 18. The user interface 18 operates via the CPU 12 using memory from the memory device 14. The user interface 18 provides interaction with interface elements on the display 16 via certain user-input structures (exampled described below), user-input peripherals (e.g., a keyboard or mouse), or a touch-sensitive implementation of the display 16. At any given time, one or more applications may be open and accessible to a user via the user interface 18 and/or displayed on the display 16 of the electronic device 10.

The various applications run on the CPU 12 in conjunction with the memory 14, the display 16, and the user interface 18. Instructions stored in the memory 14 or the CPU 12 direct the electronic device 10 to perform certain processes. As such, it should be appreciated that the instructions for carrying out such processes may represent a standalone application, as in this example embodiment, or alternatively they may represent a function of the operating system of the electronic device 10, the hardware of the CPU 12, the memory 14, or other hardware or software of the electronic device.

The network interface 20 provides wireless connectivity for the electronic device 10. The network interface 20 may include, for example, one or more network interface cards (NIC) or a network controller. In some embodiments, the network interface includes a wide-area network (WAN) interface (e.g., for connecting to a cellular data network), a local-area network (LAN) interface 30 (e.g., for connecting to a WI-FI network), and/or a personal-area network (PAN) interface (e.g., for connecting to a BLUETOOTH network).

The wired input/output (I/O) interface 22 typically includes a wired interconnection between the electronic device 10 and another electronic device for additional connectivity. The wired I/O interface 22 may be, for example, a universal serial bus (USB) port or an IEEE 1394 port (e.g., a FIREWIRE port), but may also represent a proprietary connection. Additionally, the wired I/O interface 22 may permit a connection to peripheral user-interface devices, such as a keyboard, a mouse, or headphones.

The camera 24 enables the electronic device 10 to obtain digital photographic and/or video images. In combination with optical character recognition (OCR) software, barcode-reading software, or QR-code-reading software running on the electronic device 10, the camera 24 may be used to input data from printed materials having text or barcode information. And a software application 25 stored on the memory device 14 enables a user to operably control the functionality of the camera 24, for example the shutter-speed, zoom, flash, front/rear direction, still/video, etc., to obtain, store, and display digital photographic and/or video images. In addition, the camera application 25 includes a panoramic feature operable to stitch together multiple photographs into a single panoramic photograph.

The accelerometer 26 and the gyroscope 30 sense the movement and/or orientation of the electronic device 10. Typically, one or more multiple accelerometers 26 and one or more gyroscopes 30 provide input or feedback regarding the position of the electronic device 10 to certain applications running on the CPU 12. For example, the gyroscope 30 may include a three-axis gyroscope, and the accelerometer 26 may include a three-axis accelerometer (commercially available from ST Microelectronics) that measures forces of acceleration in three dimensions. And a software application (e.g., a level application) 27 stored on the memory device 14 interprets data received from the accelerometer 26 and the gyroscope 30 to determine the position of the device 10 relative to all three (X, Y, and Z) axes. Thus, the level application 27 functions to determine whether the device 10 is level (relative to the X and Y axes) and to determine the relative angle of the device (about the Z axis). In this way, the level application 27 is capable of advanced motion sensing such as acceleration, full 3D altitude, and rotation rate. In some embodiments, the device additionally or alternatively includes one or more other position-sensing devices, such as a magnetometer, that also provides inputs to the level application 27 for determining the position/orientation of the device 10.

The vibrator 28 generates an oscillatory motion to produce a vibratory noise. The vibrator 28 is typically used as a setting to notify a user of an incoming communication, whether the speaker is turned off or on. For example, the vibrator 28 may include a vibration rotary motor spinning an eccentric cam of the type included in commercially available IPHONE 5 devices. Alternatively, the vibrator may be a linear oscillating vibrator or another type of vibration-generating device that vibrates at a frequency that causes the device 10 to rotate about its vertical axis in one direction when it is stood upright on a smooth, flat surface. And a software application (e.g., a vibrator application) 29 stored on the memory device 14 is operable to turn the vibrator 28 on and off and is typically accessed via the settings instead of being provided as a stand-alone application.

The location sensor 32 obtains information about the location and/or movement of the device 10, alone or in conjunction with the accelerometers 26. This information may enable applications to enter and/or display location-sensitive data in an innovative manner in response to a user's location and/or movement. For example, a software application 35 (referred to herein as “the ER EXPRESS app” and “the ER-reservation application”) may acquire the user's location via the location sensor 32 to provide certain ER-reservation functionality, as described in detail below. In other embodiments, the electronic device includes a dedicated location sensor for such use by the ER-reservation application 35.

Typically, the location sensor 32 includes commercially available global-positioning system (GPS) circuitry. Alternatively or additionally, the location sensor may include one or more algorithms and databases stored in the memory 14 and executed by the CPU 12 to infer location based on various observed factors, for example the location sensor may include an algorithm and database used to approximate geographic location based on the detection of local wireless networks (e.g., 802.11x, otherwise known as Wi-Fi) or nearby cellular phone towers.

And a map application 33 stored on the memory device 14 is operable to display geographical maps on the display 16, to redefine or reposition the map displayed, and to rescale the map displayed, via the user interface 18. Also, the map application 33 is operable to display the user's location, as determined by the location sensor 32, on the map displayed. Furthermore, the map application 33 is operable to display nearby (participating/member) hospitals based on location data stored for example on the memory device 14 or in a database on a wirelessly-connectable remote server computer, as described below.

Referring now to FIG. 2, the electronic device 10 includes an enclosure 40 that protects the interior components of the device 10 from physical damage and electromagnetic interference (EMI), while allow certain frequencies of electromagnetic radiation to pass through the enclosure for wireless communication. The enclosure 40 can be made of plastic, metal, composite materials, other suitable materials, or a combination thereof.

The display 16 of the electronic device 10 may include the user interface 18 in the form of a GUI, which may have a number of individual icons representing applications that can be activated. For example, the ER-reservation software application 35 can be represented by an “ER EXPRESS” icon on the device display 16. The user interface 18 on the display 16 may also include certain status indicator icons 44, which may indicate the status of various components of the device 10. For example, the status indicator icons may include a cellular-reception meter, an icon to indicate when the PAN interface 20 is active (e.g., when a BLUETOOTH network is in use), or a battery-life meter.

User-input structures 46, 48, 50, and 52 may supplement or replace the touch-sensitive input capability of the display 16 for interaction with the user interface 18. For example, the user-input structures 46, 48, 50, and 52 may include buttons, switches, a control pad, keys, knobs, a scroll wheel, or any other suitable input structures that work in conjunction with the display 16 to control functions of the device 10. Typically, the user-input structure 46 is an on/off button, the user-input structure 48 is a navigation button for navigating the user interface 18 to a home screen, the user-input structures 50 are a pair of buttons for controlling volume, and the user-input structure 52 is a sliding button that mutes the electronic device 10.

In addition, the electronic device 10 typically includes audio input and/or output structures. For example, audio structures 54 may include one or more microphones for receiving voice data from a user of the device 10. And an audio structure 56 may include and/or one or more speakers for outputting audio data, such as songs, ringtones, sound tracks associated with videos, voice data received by the device 10 over a cellular network, and so forth. In some embodiments, an audio port 58 may also enable connection of peripheral audio input and output devices, such as headsets, speakers, or microphones, for use with the device 10.

Furthermore, a wired (e.g., LIGHTNING or 30-pin dock USB) connector 59 can be provided for charging and/or connecting to other devices. And the camera 24 may be located, for example, on the back of the electronic device 10, and a front-facing camera 24 may also be included.

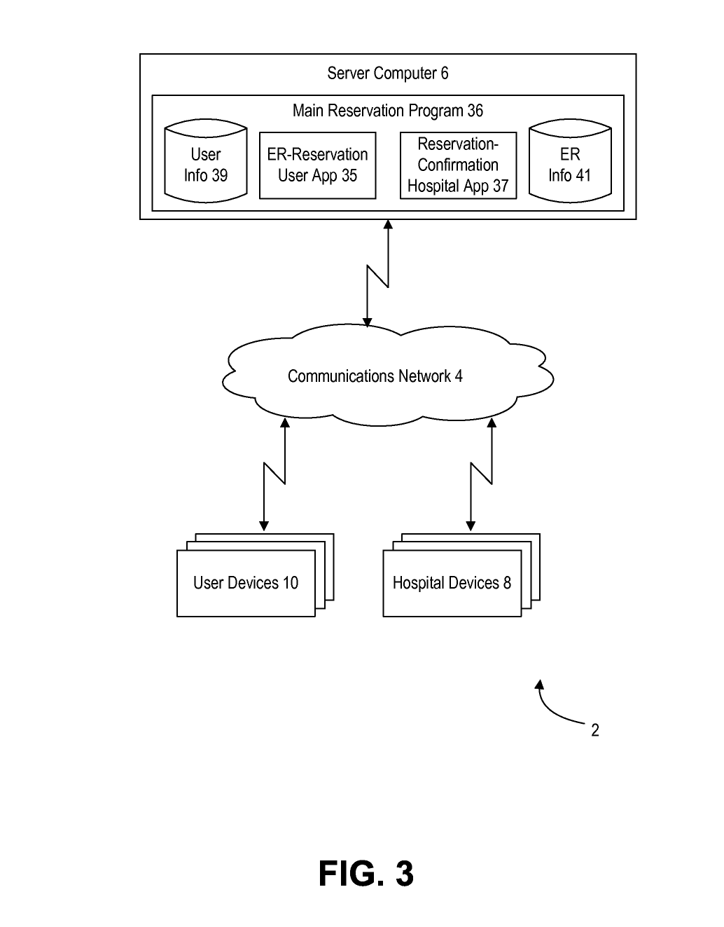

Having described certain structures and functions of the electronic device 10, additional aspects of the invention will now be described. FIG. 3 shows a system 2 for making and managing ER reservations according to an example embodiment of the present invention. The system 2 includes the user device 10 and a hospital device 8 that each communicate with a computer server 6 via a communications network 4. Typically, there are a plurality of the user devices 10 for a plurality of member users of the ER EXPRESS service, and a plurality of the hospital devices 8 for a plurality of member hospitals of the ER EXPRESS service, as depicted. (Of course, each individual member user can use a plurality of the user devices, and each individual member hospital can use a plurality of the hospital devices, if desired.)

The hospital device 8 can be a desktop-computer workstation of a conventional type well-known and commonly used in hospitals (e.g., including a processor and a non-transitory memory storage device for running a variety of hospital-related patient and admin applications), or it can be another electronic device (e.g., a laptop computer, a tablet computer, or a smart phone ala the depicted user device 10), that communicates with the user device via the server 6 (or directly in other embodiments). The computer server 6 can be provided by a bank of computers, in one or multiple locations. The computer server 6 includes a non-transitory storage device on which is saved the main program 36, which includes the ER-reservation application 35 for interaction with the users, a reservation-update application 37 for interaction with the hospitals, a user database 39 with information on the users, and a hospital database 41 with information on the hospitals (the user and ER information can be combined into a single database, if desired). And the communications network 4 can be a cellular system and/or the Internet (e.g., or both for devices that access the Internet via a cellular connection), or another communications network. Such servers and electronic devices are well-known in the art, and persons of ordinary skill in the art will understand how to adapt them to implement the system and method of the invention, so they are not detailed herein.

In one aspect, there is provided the ER-reservation application 35 that communicates with the location sensor 32 of the user device 10 to provide certain ER-reservation functionality for the user. The ER-reservation application 35 is downloaded from the server 6, stored in the memory storage 14 of the user device 10, runs on the CPU 12, and displays screens on the display 16 via the GUI 18. In the depicted embodiment, the ER-reservation application icon 35 may be selectable by a user. For example, the display 18 may serve as a touch-sensitive input device and displayed icons may be selected by touch, so that when the ER-reservation application icon 35 is selected the ER-reservation application 35 opens, as described further below.

In another typical embodiment, the ER-reservation program 35 is a mobile-optimized web application. In such embodiments, the ER-reservation program 35 is not downloaded to and stored on the user device 10, but instead is stored on the remote server computer 6 and for use is accessed by the user device via a wireless connection to the communications network 4. For example, the ER-reservation program 35 can be embedded on a hospital's website (typically, stored on a third-party server) so that users can use it while connected to and using the hospital website without disconnecting from the hospital website (while other components of the main program 36, e.g., the module for the queue process and the user database, remain on the server 6).

In another aspect, there is provided the software program 37 that communicates with the hospital electronic device 8 to provide certain related reservation-update functionality for the hospital, and as such is sometimes referred to herein as the “reservation-update application 37.” The reservation-update application 37 is downloaded from the server 6 onto and run by the hospital electronic device 8. In other typical embodiments, the reservation-update application 37 is a mobile-optimized web application. In such embodiments, the reservation-update application 37 is not downloaded to and stored on the hospital device 8, but instead is stored on the remote server computer 6 and for use is accessed by the hospital device via a wireless connection to the communications network 4. For example, the reservation-update application 37 can be embedded on a hospital's website (typically, stored on a third-party server) so that hospitals can use it while connected to and using the hospital website without disconnecting from the hospital website (while other components of the main program 36, e.g., the module for the reservation-confirmation process and the ER database, remain on the server 6).

In these and other embodiments, various aspects of the invention can include the ER-reservation process performed by the respective software application 35, the ER-reservation software application 35 itself, the user-device memory-storage device that stores the ER-reservation software application 35 for use by the electronic device 10, and/or the electronic device 10 that stores the ER-reservation software application 35. Similarly, in these and other embodiments, various aspects of the invention can include the reservation-update process performed by the respective software application 37, the reservation-update software application 37 itself, the hospital-device memory-storage device (e.g., a magnetic or optical drive) that stores the reservation-update software application 37 for use by the hospital device 8, and/or the hospital device 8 that stores the reservation-update software application 37. And in these and other embodiments, another aspect of the invention can include the server computer 6 (e.g., part of a private network for the ER EXPRESS service, part of a private network of the hospital, or remote and controlled by a third party) that stores the main reservation software program 36 for accessing and running.

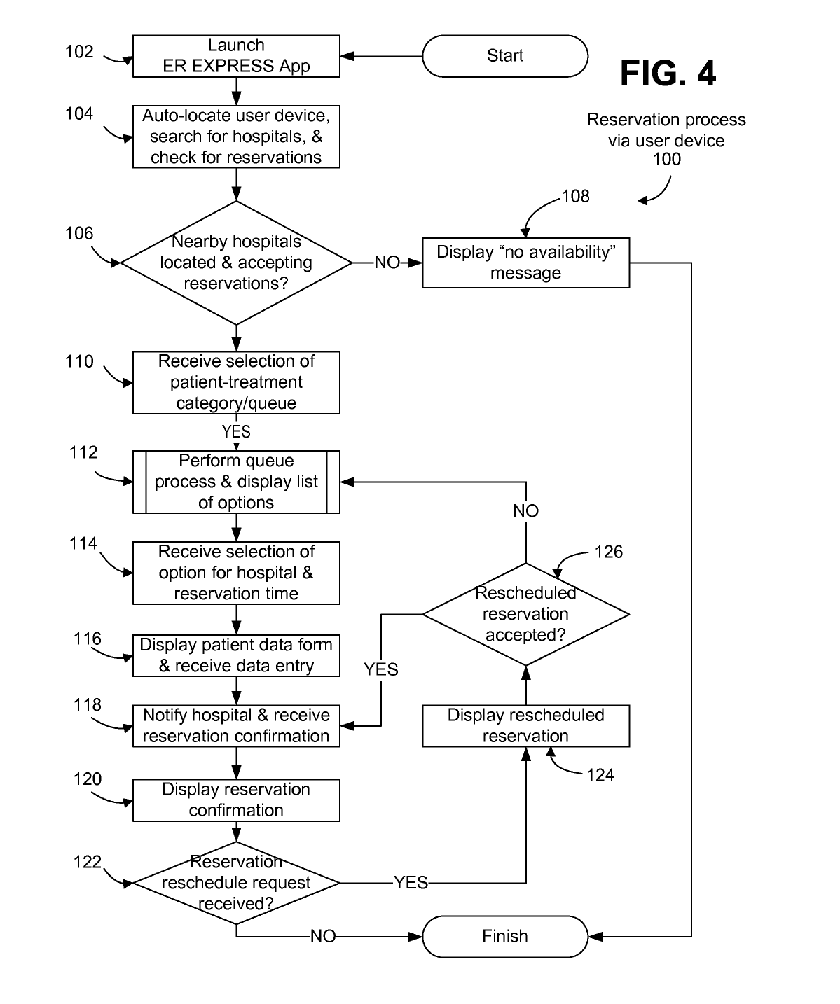

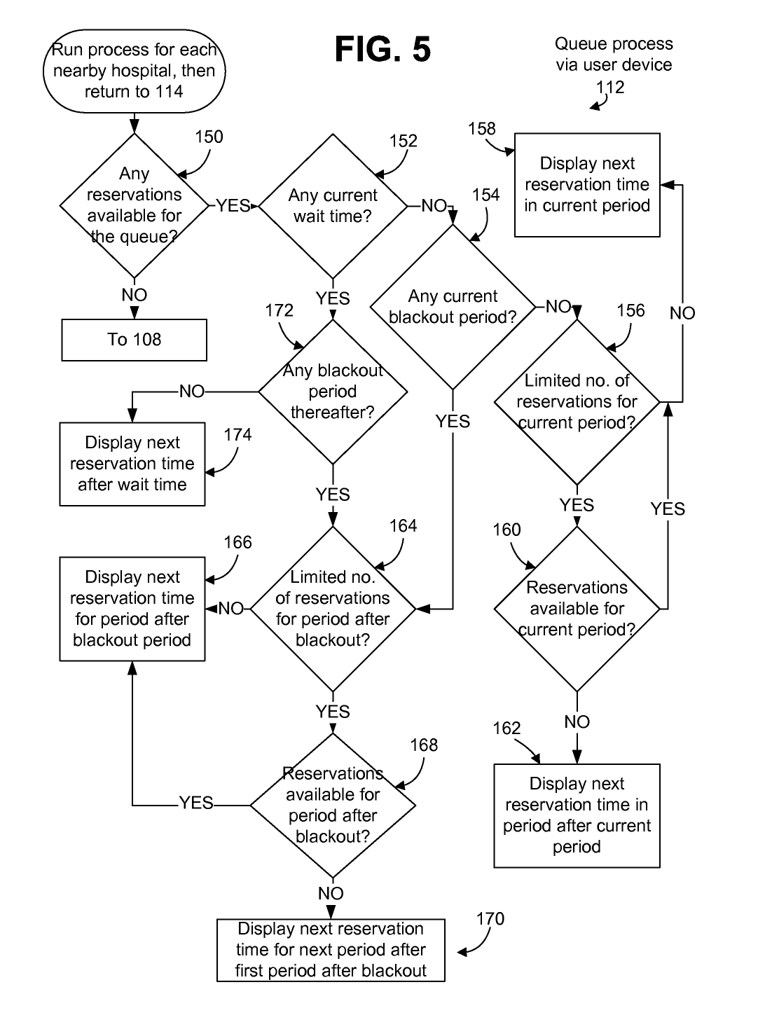

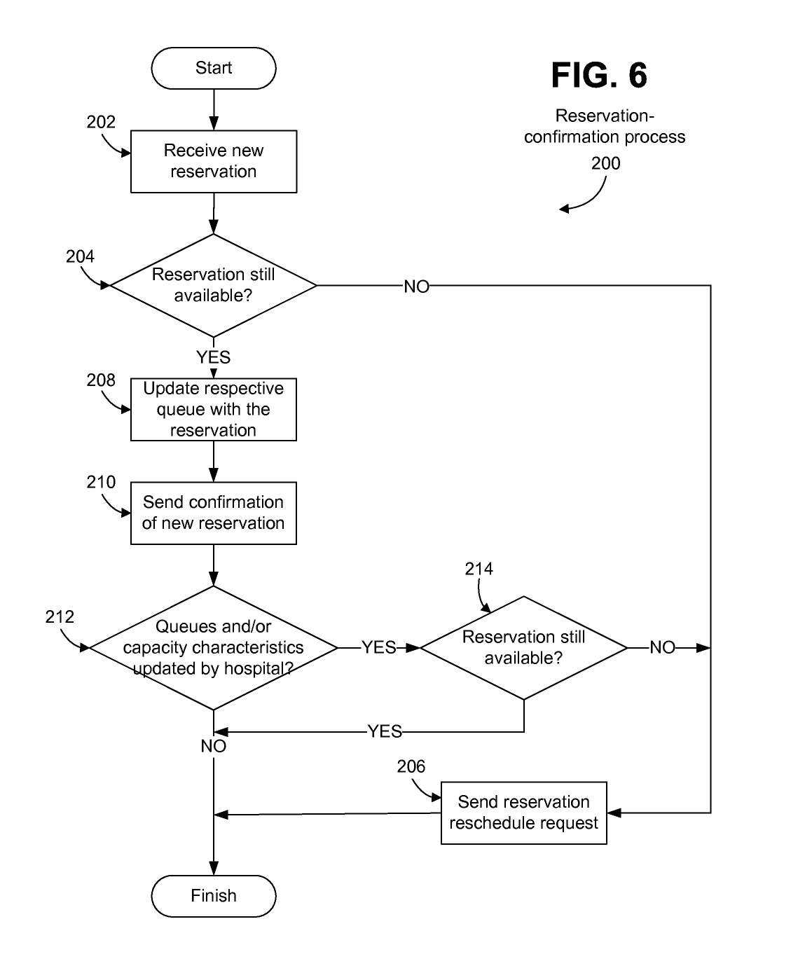

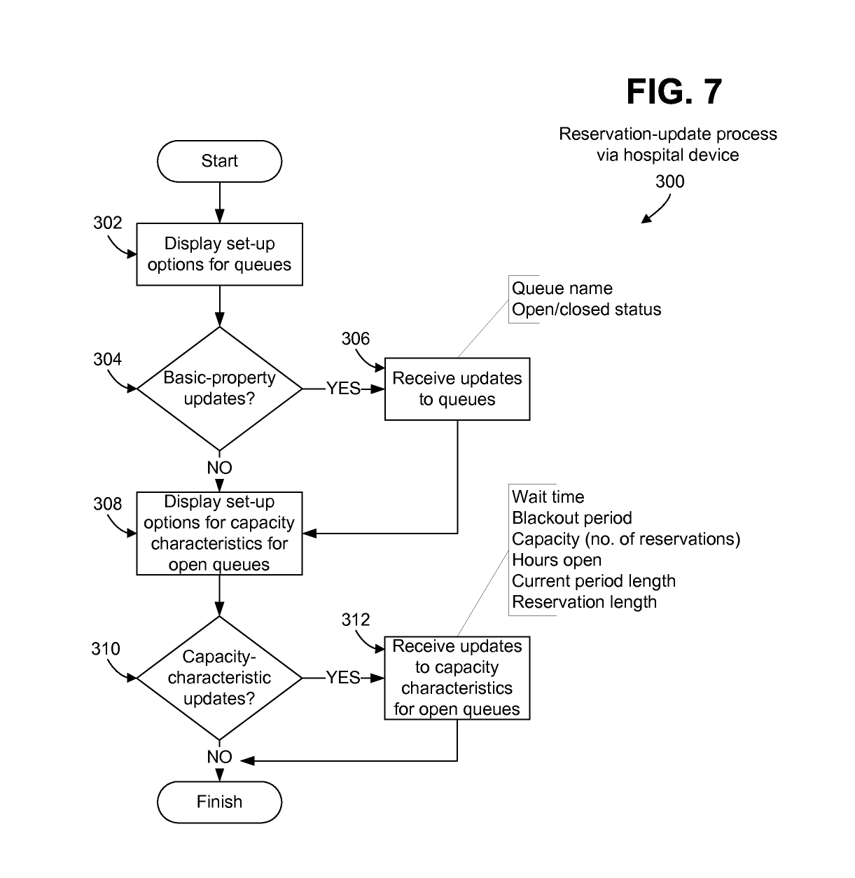

FIGS. 4-7 show process flows for ER reservations by users, reservation confirmations based on updates from the hospitals, and reservation updates by hospitals according to aspects of the present invention. As such, these flow diagrams illustrate the processes implemented by the ER-reservation application 35, the main program 36, and the reservation-update application 37. FIGS. 4-5 show an ER-reservation process 100 that is performed using one of the user electronic devices 10 and a queue process 112 that is performed by the main program 36 as a step in the ER-reservation process. FIG. 6 shows a reservation-confirmation process 200 that is performed by the main program 36 on the server 6 using updates from the hospital electronic devices 8 (or alternatively can be done by directly using one of the hospital electronic devices 8) for interaction with the ER-reservation process 100 as part of the overall system and method for ER reservations. And FIG. 7 shows a reservation-update process 300 that is performed using one of the hospital electronic devices 8 for interaction with the reservation-confirmation process 200 as part of the overall system and method for ER reservations.

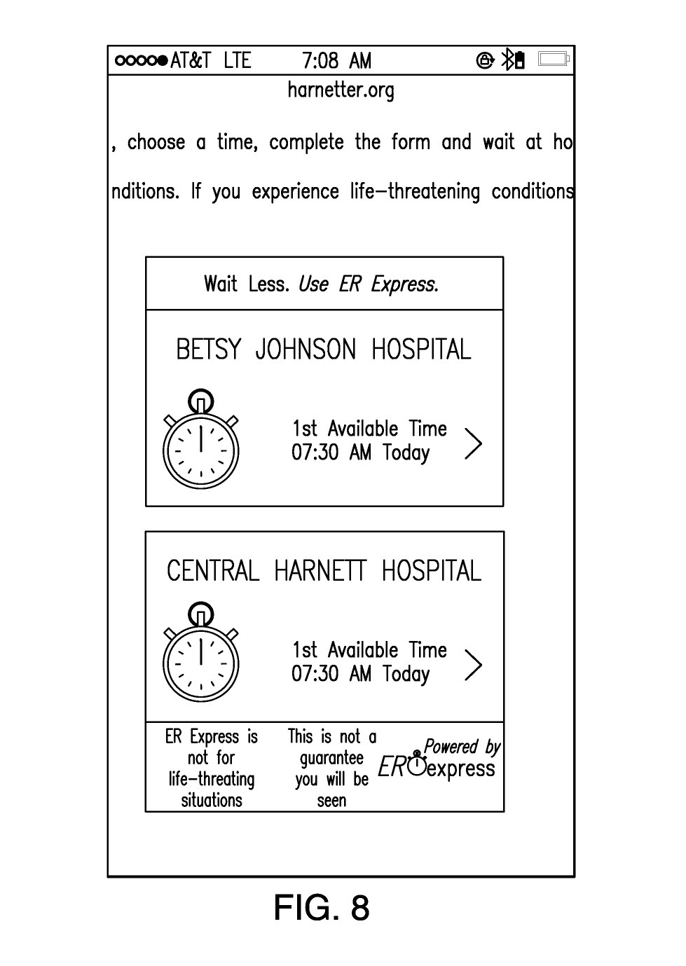

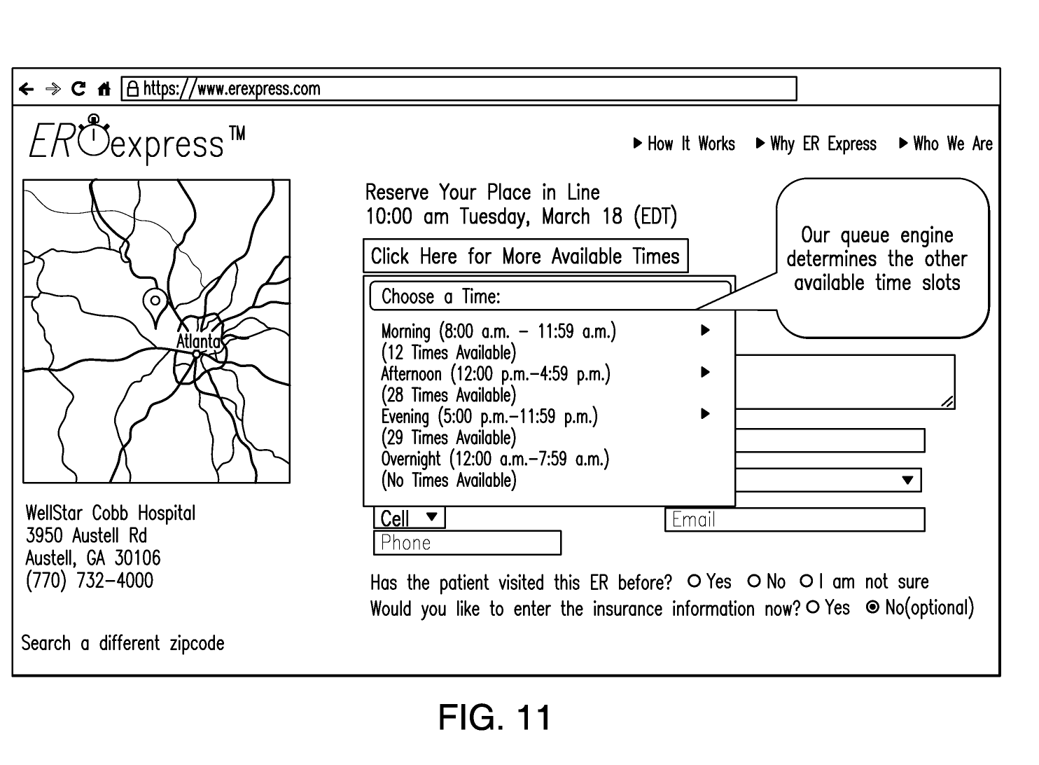

In addition, FIGS. 8-17 show screenshots displayed by the user devices 10 and the hospital devices 8 at various steps of the ER-reservation process 100 and the reservation-update process 300. These screenshots will be described below with reference to the corresponding steps of the respective processes. Except that FIG. 8 is an example user-device “home” screenshot displaying a hospital website (in this case, Harnett Hospital) with the ER-reservation application 35 embedded on it (instead of downloaded onto the user device).

As a preliminary matter before detailing the ER-reservation process 100 of FIG. 4, it should be noted that additional functionality common in conventional subscription/membership software applications is typically provided by the ER-reservation process. For example, a registration component can be included for subscribed-member users to register by entering user account information (e.g., a user name, password, and personal contact information), settings (e.g., alarms and notifications), preferences (e.g., preset a default/home user location, a default/preferred hospital, a default geographical radius for use as a search parameter in an ER search, a default maximum number of hospitals to display, a default patient-treatment category for use in a queue process), and/or the like. In addition, in some embodiments user medical information (e.g., allergies and special medical conditions) can be entered. This user-related information can be stored in a database 39 located for example on the server computer 6 or elsewhere. Persons of ordinary skill in the art understand how to make and use such additional conventional software components, so they are not detailed herein for brevity.

To perform the ER-reservation process 100 of FIG. 4, at step 102 the user launches the application 35 on a user device 10. Then at step 104 the application 35 receives a hospital search parameter(s) from the user device 10 and conducts an ER search to identify hospitals that meet the hospital search parameter(s). In addition, for any identified hospitals, the application 35 typically checks (e.g., with the main program 36 or directly the hospital devices 8), to determine whether they are accepting any new ER reservations. In some embodiments, the accepting-reservations check is eliminated from this step 104 and included in the queue process 112 described below.

In typical embodiments, the hospital search parameter(s) is related to the location of the user device 10, and the application 35 receives user-device location information from the user device. In the depicted ER-reservation process 100, for example, the application 35 performs an auto-locate process to determine the location of the user device 10 (and thus the user), then conducts an ER search to identify hospitals near the user-device location based on the hospital search parameter(s). In particular, the user device 10 (and thus the user) is auto-located at step 104 by using the location sensor 32 of the user device to obtain information identifying the geographic location of the user device. And the ER-reservation application 35 interfaces with the location sensor 32 of the user device 10 to receive the user-device location information from it. Such auto-locate processes are well-known in the field of mobile applications, and are understood by persons of ordinary skill in the field, so details of the process are not included herein.

In other embodiments, at this step the ER-reservation process includes displaying a field on the user device 10 for the user to input location-identifying information (e.g., an address) to manually identify the user location information. This can be of use for example when it is desired to search for hospitals based on a user location that is not the current location of the device user but instead is a future location of the user or a location of another user (when searching on behalf of a sick or injured person who is not at the same place as the user). This can also be of use in the case of users with electronics devices that do not have location-sensing capability (e.g., some desktop computers). In some such other embodiments, this manual-location feature is in addition to the auto-location feature such that a user-device location sensor is still needed for full functionality, and in other such embodiments this manual location is a substitute such that a user-device location sensor is not needed or used.

And in other embodiments, at this step the process conducts the ER search and the accepting-reservations check without knowing the user location. In such embodiments, the ER search is based only on ER search parameters (e.g., a county, a city, or a zip code to search) that define a geographical search area independent of (without having to identify) the user location (which thus may or may not be within the geographical search area).

The hospital search parameters used at step 104 can include for example a desired geographical search area (e.g., defined by a radius such as 50 miles from the user location, a maximum travel time for example by driving, a metropolitan area, a county, a city, or a zip code), inclusion or exclusion of certain facility types (e.g., hospital ERs, urgent-care clinics, or pediatric care), a maximum number of hospitals to display, and/or other criteria. In some embodiments the process includes conducting the ER search based on preset parameters (e.g., default/preferred parameters saved in the settings/preferences) without requesting input from the user. And in some embodiments the process includes displaying a field on the user device 10 providing the user the opportunity to confirm preset search parameters or input custom search parameters.

Whether a hospital is “near” or “nearby” is defined based on the hospital search parameters. For example, for a single hospital search parameter of a 30-mile radius of the user device 10, a hospital that is 30 miles or less away from the user device is nearby and one that is farther is not.

The ER search conducted at step 104 can include accessing a database 41 of hospital locations and then looking up the hospital locations that fit the hospital search parameters. The ER location database 41 can be stored on the server computer 6 and maintained by the provider of the ER EXPRESS service, or stored elsewhere and/or maintained by others. For example, location information for each hospital can be populated into the ER location database 41 when the hospital registers as a participating hospital member, as described below.

If at step 106 it is determined that there are no nearby hospitals accepting any new ER reservations, then at step 108 a message to that effect is displayed on the user device 10 to the user. At that point, the process 100 can be exited or the user can modify the search parameters (e.g., expand the geographical search area).

If at step 106 it is determined that there are nearby hospitals accepting any new ER reservations, then at step 110 the user is prompted to enter a patient-treatment category (type) search parameter(s) (e.g., from a menu of preset options). The patient-treatment category can be, for example, adult, child/pediatric, urgent care (lower acuity), direct admit (higher acuity), walk-in, worker's comp, or another category defined by the participating member hospitals or the ER EXPRESS service provider. Alternatively, in some embodiments the process can include an option for the user to type in custom (user-defined) patient-treatment category search criteria, and in some such embodiments the process can including searching a database (e.g., on the server 6) of pre-defined categories and displaying those that substantially match the spelling of the custom category entered by the user. The patient-treatment categories are also referred to herein as “queues,” as each category/queue defines a type of patient treatment that may or may not be available at all member hospitals.

Upon receipt of the entered patient-treatment category/queue selection at step 110, a queue process is performed at step 112 to recommend (identify and display) the best options for hospital and reservation time for the patient given the hospital search parameters and the treatment category (queue). Details of the queue process 112 are described below with reference to FIG. 5. In other embodiments, the ER search is not conducted until the queue selection is entered so that the initial ER search can be also based on the entered patient-treatment category parameter and can thus be included as a step in the queue process.

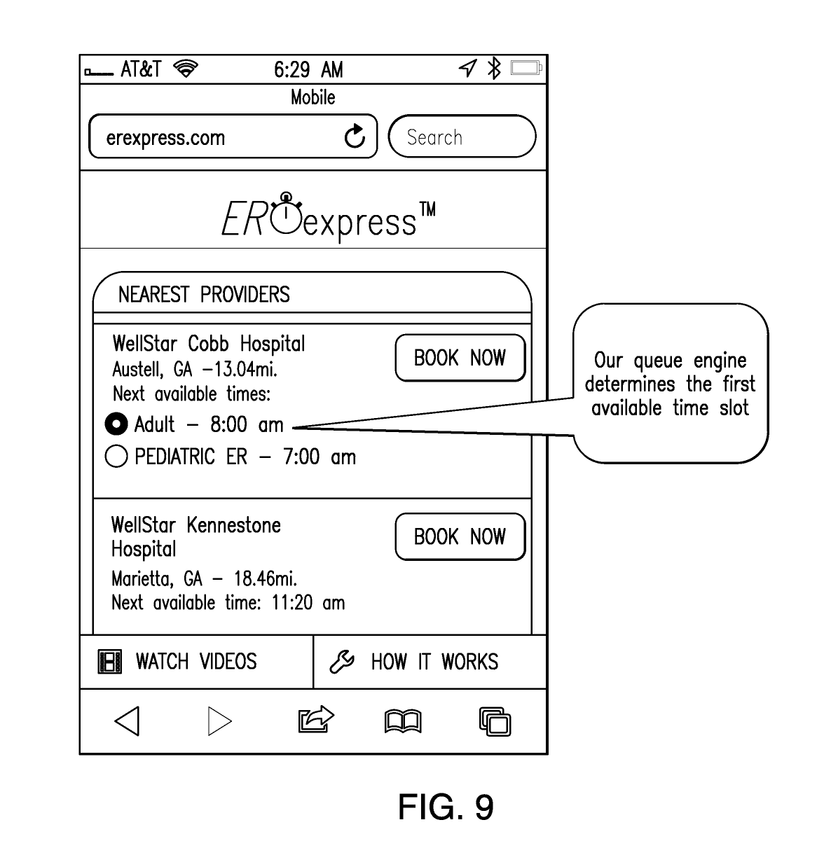

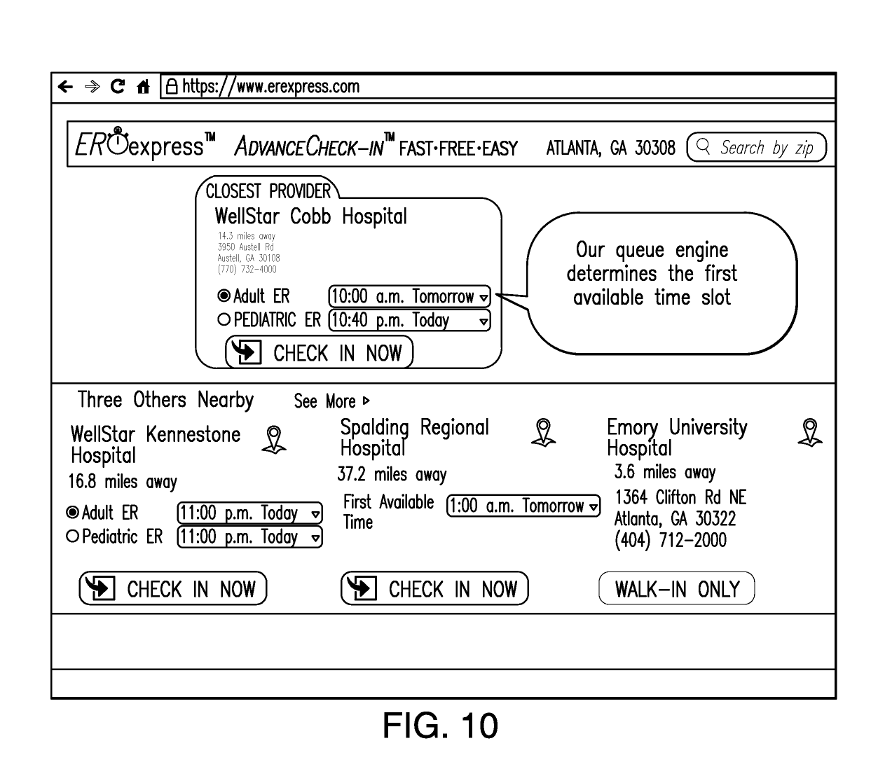

Example user-device “reservation options” screenshots displaying example hospital and reservation-time options are shown in FIG. 9 (for the smart-phone user device 10) and in FIG. 10 (for an alternative desktop-computer user device). In these example screenshots, the queue process 112 displays the first available reservation time for the best-recommended (nearest) hospital out of all of the hospitals within the geographic search area (as returned by the ER search).

Next, the user enters a selection of an option of a hospital and reservation time at step 114. Then a patient data form (with data-entry fields) is displayed and the patient enters relevant personal and medical data at step 116. For example, upon the user selecting the most-recommended (nearest) hospital displayed on the “reservation options” screen of FIG. 10, an example user-device “reservation details” screenshot such as that of MG. 11 displays additional information about the selected hospital (e.g., including a map identifying the hospital location using the map application 33), a list of other available reservation times for that hospital (e.g., in a drop-down menu, as depicted), and the patient data form. Upon receipt of the required information to complete the patient data form, notice of the reservation request is sent to the hospital device 8 and a confirmation is received from the hospital device at step 118. And a confirmation (e.g., a confirmation number) is then displayed on the user device 10 to the user at step 120 to confirm the reservation has been made.

If at some point after this, but before the reservation time, the reservation needs to be rescheduled as determined by receiving a reschedule request at step 122 (see also FIG. 6), then at step 124 a notice to this effect is displayed to the user on the user device 10. If the user accepts the rescheduled reservation (e.g., the next-best available reservation as previously determined) at step 126, then the process returns to step 118 to confirm with the hospital. If the user declines the rescheduled reservation at step 126, then the process returns to and repeats the queue process at step 112 to find another reservation for the patient. And if no reschedule request is received at step 122, then the ER reservation process is completed and the user simply awaits the scheduled reservation time. In some embodiments, at step 124 a rescheduled reservation is not displayed to the user, but instead there is displayed a choice to either request at step 126 rerunning the queue process 100 (to anew find the best available reservation) by returning to step 112 or request canceling/exiting the entire process.

FIG. 5 shows details of the innovative queue process performed at step 112 of the ER reservation process 100 of FIG. 4. As mentioned above, the queue process 112 recommends (identifies and displays) the best options for hospital and reservation time for the patient given the user's location and treatment category (queue). The queue process 112 is performed for a first hospital (e.g., of the hospitals within the geographic search area, the one closest to the user device 10), and is then repeated for additional hospitals within the geographic search area (e.g., sequentially for each of the next-closest hospitals, optionally up to a preset maximum number of hospitals).

The “best” or “recommended” option can be defined to mean the first available reservation time at the closest hospital, the first available reservation time at any hospital within the geographic search area (e.g., to obtain any earlier reservation time even though at a farther-away hospital), an available reservation time at a hospital within the geographic search area that results in the earliest available reservation time that can be met (e.g., to obtain the absolute earliest possible makeable reservation) or another predefined best-ranking scheme. In some embodiments, the ER reservation process 100 displays on the user device 10 options for sorting by different best-ranking schemes.

The “closest” hospital can be defined to mean the shortest distance from the user device 10. The shortest distance can be based on mapped street routes in the map application 33 or the linear (“as the crow flies”) distance. In some embodiments, the “closest” hospital can be defined to mean the shortest travel time from the user device 10, for example by using the map application 33 and based on speed-limit information for different street routes and/or current traffic information. Or the “closest” hospital can be defined based on another predefined close-ranking scheme, for example based on walking routes, bicycle routes, train routes, or the like. In some embodiments, the ER reservation process 100 displays on the user device 10 options for sorting by different close-ranking schemes.

At step 150, the process 112 checks with the first hospital (e.g., via the main program 36 or directly the first hospital device 6) to determine if it has any available reservations for the queue selected by the user. If not, then the process 112 starts over for the next hospital, and if determined by repeating the process that there are no nearby hospitals with any available reservations for the queue, then the process 112 returns to step 108 to display a message to that effect on the user device 10, after which the user can exit or modify the search parameters.

If there are reservations available in the selected queue at the subject hospital (the first one or a next one), then at step 152 the process 112 checks with the subject hospital to determine if there is currently a wait time for the queue. If not, then at step 154 the process 112 checks with the subject hospital device to determine if the current time period is a blackout period for the queue. (Blackout periods allow ER staff to block specified times of day where no reservation slots are available; these periods act as an override.) If not, then at step 156 the process 112 checks with the subject hospital to determine if there is currently a fulfilled limit on the number of reservations (i.e., the “throttle”) for the current time period (e.g., one hour) for the queue. If not, then at step 158 the process 112 gets the next available reservation time in the current time period for the queue at the subject hospital and displays that option to the user on the user device 10.

If at step 156 it is determined that there is currently a fulfilled limit on the number of reservations for the current time period for the queue at the subject hospital, then at step 160 the process checks with the subject hospital to determine if there are any remaining reservations available for the current period for the queue. If there are, then the process 112 returns to step 158 to display this option to the user device 10. But if not, then at step 162 the process 112 gets the next available reservation time in the subsequent time period (e.g., one hour) after the current time period for the queue at the subject hospital and displays that option to the user on the user device 10.

If at step 154 it is determined that the current time period is a blackout period, then at step 164 the process 112 checks with the subject hospital to determine if there is a fulfilled limit on the number of reservations for the subsequent time period (e.g., one hour) after the blackout period for the queue. If not, then at step 166 the process 112 gets the next available reservation time for the subsequent time period after the blackout period and displays that option to the user on the user device 10. But if at step 164 it is determined that there is currently a fulfilled limit on the number of reservations for the subsequent period after the blackout period for the queue, then at step 168 the process 112 checks with the subject hospital to determine if there are any remaining reservations available for the subsequent period (after the blackout period) for the queue. If there are, then the process goes to step 166 to get the next available reservation time for the subsequent time period after the blackout period and displays that option to the user on the user device 10. But if not, then at step 170 the process 112 gets the next available reservation time for the next subsequent period (after the first period after the blackout period) and displays that option to the user on the user device 10.

If at step 152 it is determined that there is currently a wait time, then at step 172 the process 112 checks with the subject hospital to determine if the subsequent time period after the wait time for the queue is a blackout period. If it is, then the process 112 goes to step 164 to check on any fulfilled limit on the number of reservations and the available reservations for the subsequent period after the blackout period. If not, then at step 174 the process 112 gets the next available reservation time for the subsequent period after the wait time and displays that option to the user on the user device 10.

In this way, upon completion the queue process 112 displays to the user via the user device 10 a list of options for hospitals and reservations times. And the listed hospitals and reservation times are can be ranked, for example, with a best option recommended by being listed first. For example, the process 112 can identify typical or current travel times to the nearby hospitals and, based on reservation availability for the selected queue, determine which hospital option gets the user the earliest reservation time. So the process 112 makes recommendations of where and when to go.

As an example, a child might slip and fall, with a parent suspecting a possibly broken bone, and decide that an ER visit is appropriate. The parent could simply drive to the nearest hospital 5 minutes away and wait for unknown hours and hours for the child to be examined. But using the ER-reservation service, the parent could find out in advance that the nearest hospital has a long wait time and a lengthy blackout period after that, that the next-closest hospital at 12 minutes away does not currently have a queue open for ER care for children (e.g., no pediatrician currently on duty), and that the third-nearest hospital has a reservation available in the current hour and is only 10 minutes farther away than the closest hospital. So the ER-reservation service could display these options, with the third-closest hospital recommended and thus ranked above the closest hospital, because its reservation time is the soonest one that can be met (the hospital can be reached in time so the reservation is not missed).

In other embodiments, the queue process includes other capacity characteristics, for example less than these three, additional ones, or other combination of some or all of these and others. The capacity characteristics can include any factor that impacts the treatment capacity of the ER, including hours of operation, lengths of time periods, lengths of reservation, and even open/closed status and queue type (“basic control properties” discussed separately herein).

Turning now to the reservation-confirmation process 200, FIG. 6 shows details of the process performed by the main program 36 on the server 6 for interaction with the ER reservation process 100 at step 118 of FIG. 5. Alternatively, the reservation-confirmation process 200 can be performed by the hospital devices 8, which in that case communicate with the user devices directly or via the main program 36. The reservation-confirmation process 200 can be a completely automated process, or alternatively it can be carried out in combination with manual input from a hospital staff person using the respective hospital device 8.

As a preliminary matter before detailing the reservation-confirmation process 200 of FIG. 6, it should be noted that additional functionality common in conventional subscription/membership software applications is typically provided. For example, a registration component can be included for subscribed-member hospitals to register by entering user hospital information (e.g., a user name, password, contact information, and location information), settings (e.g., alarms and notifications), preferences (e.g., preset default blackout periods, or limits on the number of reservations per time period), and/or the like. In addition, the hospital can enter ER schedule information at that time or later (in the reservation-update process 300), and then update the ER schedule information later (in the reservation-update process 300). This hospital-related information can be stored in a database 41 located for example on the server computer 6 or elsewhere. As such, the hospital location information mentioned above with reference to the ER search at step 104 of the ER-reservation process 100 can be populated into the ER information database 41 when the hospital registers as a participating member hospital of the ER EXPRESS service. Persons of ordinary skill in the art understand how to make and use such additional conventional software components, so they are not detailed herein for brevity.

The reservation-confirmation process 200 includes at step 202 receiving a new reservation request and at step 204 checking with the hospital device 8 whether that reservation is still available. For example, if there was some delay in the user selecting a hospital and reservation option after they were displayed on the user device 10, and in the interim another user confirmed that reservation, then that reservation would no longer be available. In that event, at step 206 a reservation reschedule request would be sent to the user device 10 for interaction with the ER-reservation process 100 at step 122.

If the reservation is still available at step 204, then at step 208 the respective queue (e.g., pediatric) is updated to show that reservation as taken and thus no longer available to other users. And at step 210 a reservation confirmation is sent to the user device 10.

If sometime afterward conditions at the subject hospital have changed and affected its reservation scheduling (for better or worse), the process 200 enables the hospital to update its ER schedule information (e.g., on the server 6 and/or the hospital device 8) to reflect that. So if at step 212 the process 200 determines that any of the queues and/or any of the capacity characteristics (e.g., wait time, blackout period, or limit on number of reservations per time period) have been updated by the hospital, then at step 214 it checks if the reservation is still available in light of the updates. It not, then the process 200 goes to step 206 to run through the rescheduling process described above and repeat the queue process 100 as needed. And if it is, then the reservation remains intact (the process 200 is finished by using the reservation).

Turning now to the reservation-update process 300, FIG. 7 shows details of the process performed by a hospital device 8 (typically via manual input from a hospital staff person) at step 212 of the process 200 of FIG. 6. The update process 300 includes at step 302 displaying on the hospital device 8 the queues and an option for the hospital staff person to enter updates to the basic control properties of the queues via a secure, internal admin panel. If at step 304 it is determined that any basic properties of any queues have been updated, then at step 306 the queue updates are saved. For example, such updates can include the names of the queues and their status (e.g., open or closed). (The open/closed status is the property checked at step 150 of the queue process 112.) So as the situation in the ER changes over time, the hospital has the flexibility to for example open an extra queue for a particular patient-treatment category if that is needed, or change a queue from one patient-treatment category to another to meet the treatment needs of its patients and schedule them more efficiently. As noted above, these basic control properties can be considered “capacity characteristics” of the treatment categories/queues (so updating them for better or worse prompts a reschedule request).

If there are no queue updates at step 304, or if there are and they have been updated at 306, then at step 308 the hospital device displays options for updating the capacity characteristics of the queues. The capacity characteristic can include, for example, a wait time, a blackout period, a limit on the number of reservations (i.e., a “throttle”), hours of operation, and/or other schedule characteristics that impact the treatment capacity of the ER.

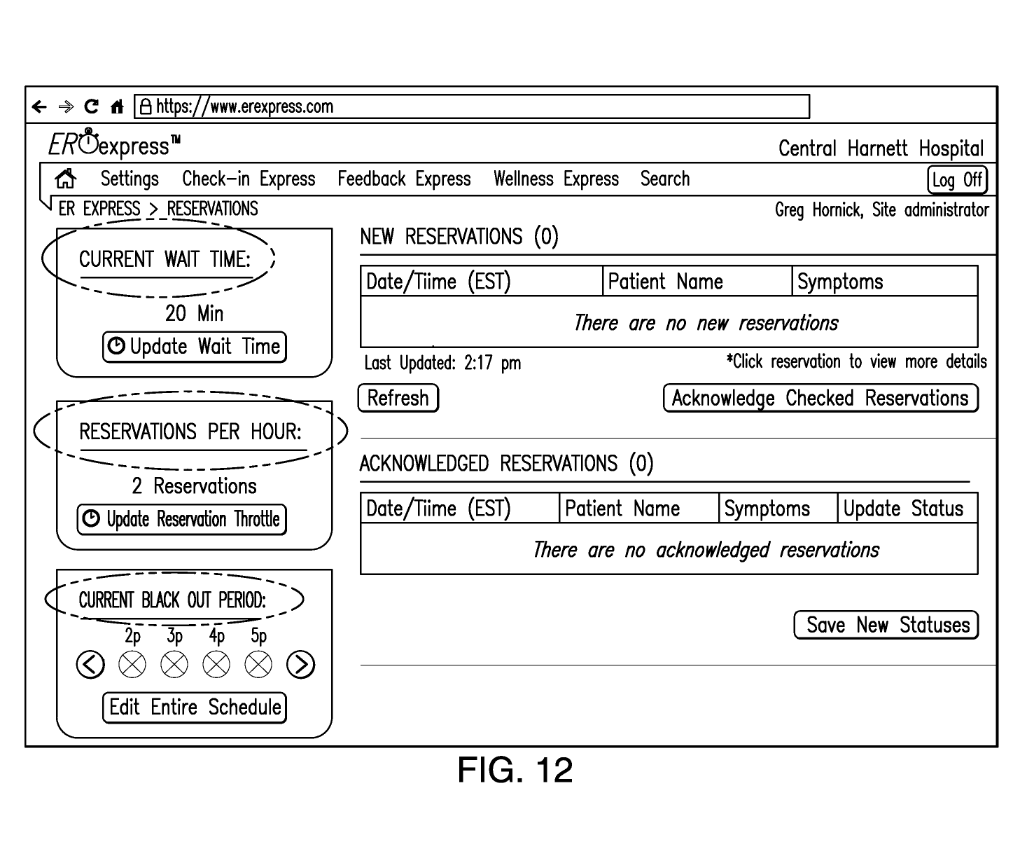

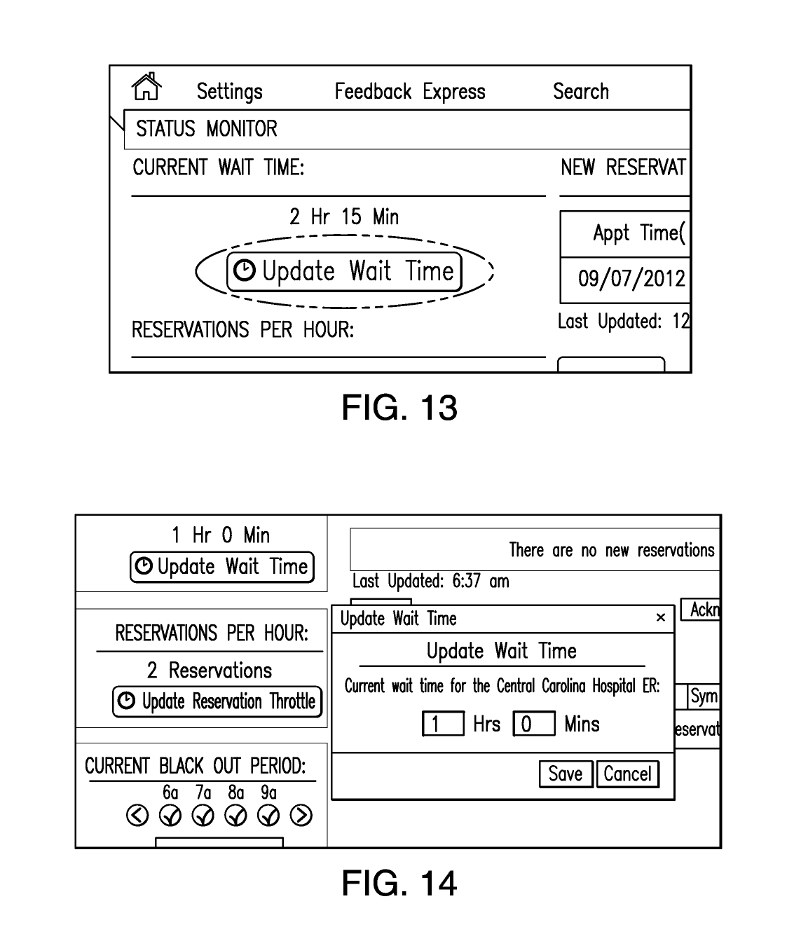

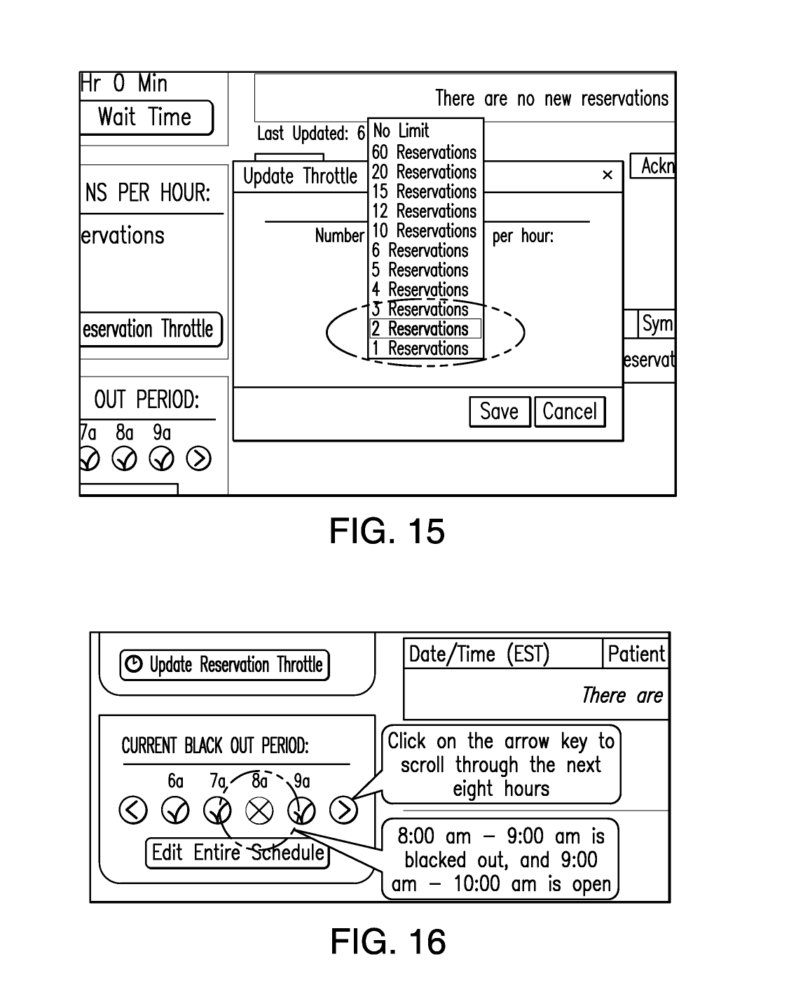

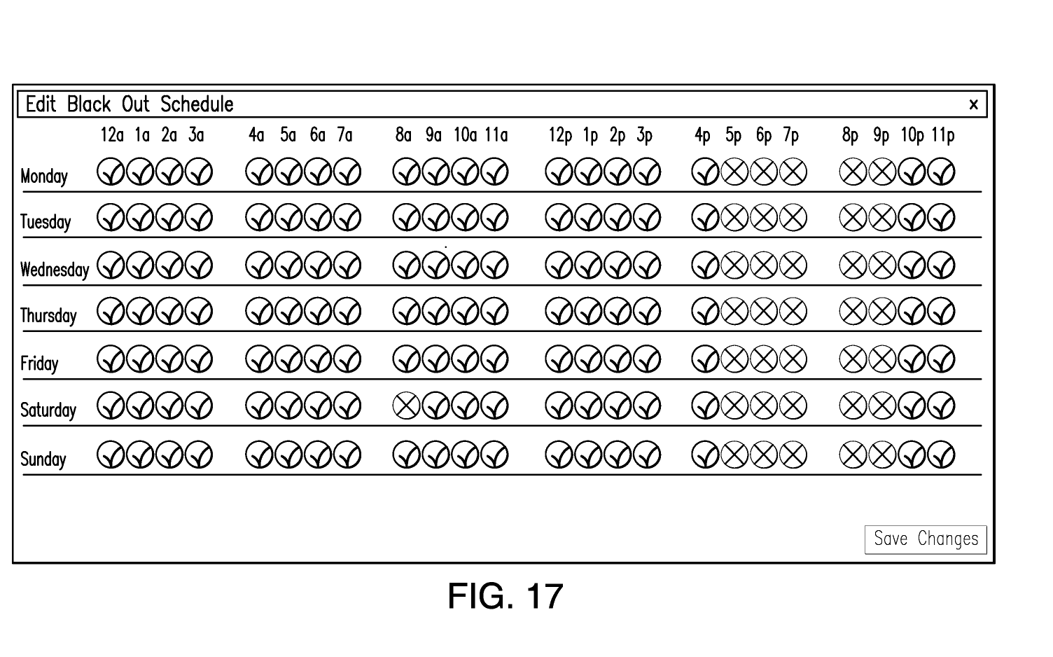

For example, FIG. 12 shows an example “reservations” screen displayed on the respective hospital device 8 with designated areas for three capacity characteristics (i.e., wait time, throttle, and blackout period) for a selected one of the queues. Each of the capacity-characteristic areas displays an indicia (e.g., name and/or icon) and current setting for the corresponding capacity characteristic, and a button for updating the current setting. Upon activating the “update” button for the wait-time capacity characteristic (see also FIG. 13) on the reservations screen, a “wait-time control” screen is displayed that can be interfaced with by the hospital staff person using the hospital device 8 (see FIG. 14) to update the wait time. Similarly, upon activating the “update” button for the throttle capacity characteristic on the reservations screen, a “throttle control” screen is displayed that can be interfaced with by the hospital staff person using the hospital device 8 (see FIG. 15) to update the throttle. In addition, the blackout capacity characteristic can be updated directly on the reservations screen (see FIG. 16) by the hospital staff person using the hospital device 8. And upon activating the “edit entire schedule” button on the blackout area of the reservations screen, a “blackout control” screen is displayed that can be interfaced with by the hospital staff person using the hospital device 8 (see FIG. 17) to update blackout periods over an extended period such as an entire week.

If at step 310 it is determined that any capacity characteristics have been updated by the hospital device 8, then at step 312 the capacity-characteristic updates are saved. If there are no capacity-characteristic updates at step 310, or if there are and they have been updated at 312, then the update process 300 is complete.

Continuing with the ER pediatric-care example described above with respect to FIG. 5, sometime after the reservation has been confirmed, the third-closest hospital might need to update its capacity characteristics to reflect that it now has a wait time (e.g., perhaps there was a walk-in patient with an immediate life-threatening condition that pushed back everyone else). So upon the hospital staff person using the hospital device 8 to update the wait-time control screen for the corresponding queue using the reservation-update process 300 at step 312, at step 206 the reservation-confirmation process 200 would send a reschedule request to the user device 10. Then the user would have the opportunity to accept a rescheduled-later reservation time or decline and check anew for the current best option of hospital and reservation using the ER-reservation process 100. So if the second-closest hospital had a pediatrician arrive for duty and has no current wait time or blackout period, then the user could reschedule a sooner reservation time at that hospital and get the child examined sooner.

Having described core details of aspects of the invention, additional supporting details will now be provided. In typical embodiments, the ER EXPRESS service is implemented in a suite of software-based services that improve the patient experience, before, during, and after they visit the ER. This suite can include modules that are sometimes referred to as ADVANCE CHECK-IN, ER PASSPORT, CHECK-IN EXPRESS/VIRTUAL LOBBY, FEEDBACK EXPRESS, AFTERCARE EXPRESS, and WELLNESS EXPRESS. It will be understood that in some embodiments these modules can be provided individually or in any combination as may be beneficial for a given application.

The ADVANCE CHECK-IN module (also referred to herein as the ER reservation application 35 above) is described above with reference to FIGS. 1-5 and 8-11. Generally, this module enables patients to check-in and pick a time slot online, then hold their place in line while they do some (or most) of their waiting at home. Patient users provide their present or desired location (e.g., by using the auto-locating feature which uses the location sensor 32 of the user device 10, or by entering user location information such as address or zip code) and selected ER search parameters (e.g., a radius defining a geographical search area). Then an innovative queue process/engine recommends (identifies and displays) nearby member hospitals with available reservation times, for example ranking them to indicate where and when a patient should go given the care needed. The patient can thereby view real-time appointment times and reserve their place in line. Rather than spend hours awaiting treatment in a crowded ER waiting room, patients can wait in the comfort of their own homes.

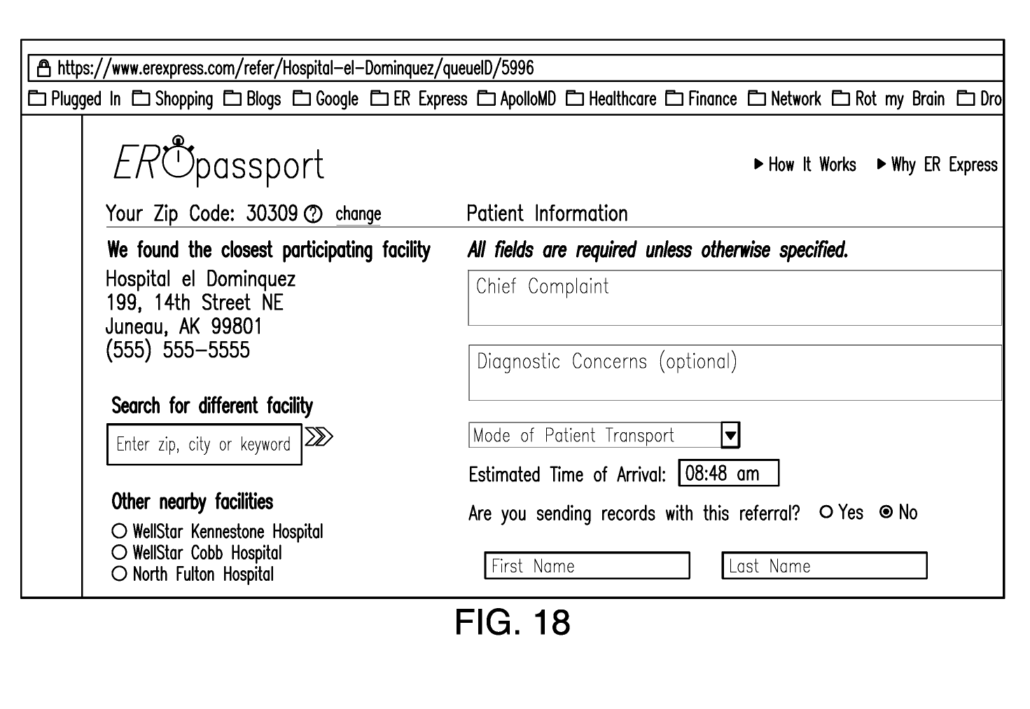

The ER PASSPORT module enables advance notification from a community physician office to improve the referral process from physician clinic to ER and provide expedited service/care. This module is used by the referring healthcare provider (via a referrer electronic device connected to the main program 36 on the server 6 via a wired or wireless connection), not the patient, and is particularly advantageous in cases in which the patient is seriously sick or injured (e.g., upon diagnosis by a primary-care physician of a diabetic foot ulcer needing immediate surgery). As such, the auto-locating feature of the referrer/user electronic device functions to geo-locate the referring healthcare professional user and recommends the closest hospital ER accepting referrals. A referral screen (see FIG. 18) is generated by this module, for example from a home screen of this module, and can be auto-populated with patient information stored in the referrer/user electronic device (including a connected server of the referring healthcare provider's IT network/system).

In addition, the ER PASSPORT module includes an innovative queue process that is functionally substantially the same as the queue process 112 of the ER-reservation process 100. As such, this referrer/user queue process recommends (identifies and displays) where and when a patient should go based on the care needed, and persons of ordinary skill in the art will readily understand how to modify the ADVANCE CHECK-IN module to implement his module. Accordingly, claims herein referring to an ER-reservation invention including a queue feature expressly cover this module and its use.

The CHECK-IN EXPRESS/VIRTUAL LOBBY module enables walk-in patient users to check in from a mobile user device (saving hospital personnel the time of entering the patient data) and get a message (e.g., SMS text) when it's time to report to the ER front desk (e.g., when an exam room is ready). So the patient can go to the hospital cafeteria to wait, instead of sitting in the ER waiting room, without fear of being called and missing that spot in line. Because this module is for walk-ins, the auto-locate feature is not needed.

In addition, the CHECK-IN EXPRESS/VIRTUAL LOBBY module includes an innovative queue process that is functionally substantially the same as the queue process 112 of the ER-reservation process 100. As such, this walk-in queue process recommends (identifies and displays) where and when a patient should go based on the care needed, and persons of ordinary skill in the art will readily understand how to modify the ADVANCE CHECK-IN module to implement his module. Accordingly, claims herein referring to an ER-reservation invention including a queue feature expressly cover this module and its use.

The FEEDBACK EXPRESS module enables patients to provide feedback by text message to alert ER staff of service recovery opportunities. The AFTERCARE EXPRESS module provides access to web-based videos explaining aftercare instructions to discharged ER patients, e.g., delivered bedside via tablet computers or other mobile electronic devices. And the WELLNESS EXPRESS module provides interactive videos, surveys, and games to engage patients, e.g., delivered bedside via tablet computers or other mobile electronic devices.

The ER EXPRESS service thereby provides a number of advantages and benefits. For patients/users, these typically include no cost to use, less hassle/more convenience, a shorter wait time, and increased control of the ER experience. For care providers/hospitals, these typically include quick implementation (e.g., “want it” to “using it” in four weeks), easy (all or most of the work is done for them), enhanced patient satisfaction, and optionally in the cloud (nothing to install or maintain). In summary, overall benefits typically include (1) for patient users, fast, easy, and free to use, in particular, all of the services are free, and these include fast, convenient tools to improve theft experience; (2) for users and/or hospitals, reduced congestion, enhanced patient satisfaction, and the ability to drive market share for hospitals; (3) for users and/or hospitals, the software is completely HIPAA-compliant and uses secure sockets layer (SSL), with extensive documentation provided; (4) for hospitals, front-line ER staff have full control over how to deploy the services, and programs can be ramped up slowly with low disruption to existing clinical and operational processes; and (5) for hospitals, the software is easy and quick (e.g., 4-5 weeks) to implement, with the software optionally being fully web-based with nothing to install, and with all of the solutions available to be implemented on an a la carte basis.

It is to be understood that this invention is not limited to the specific devices, methods, conditions, or parameters described and/or shown herein, and that the terminology used herein is for the purpose of describing particular embodiments by way of example only. Thus, the terminology is intended to be broadly construed and is not intended to be unnecessarily limiting of the claimed invention. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the invention has been shown and described in exemplary forms, it will be apparent to those skilled in the art that many modifications, additions, and deletions can be made therein without departing from the spirit and scope of the invention as defined by the following claims.

What is claimed is:

1. A computer-implemented method for booking an ER reservation with a user electronic device, comprising:
receiving at least one hospital search parameter from the user device;
identifying hospitals that meet the hospital search parameter;
receiving at least one treatment-category parameter from the user device;
performing a queue process that includes identifying, at each of the identified hospitals, an available reservation that meets the treatment-category parameter based on one or more capacity characteristics of the identified hospitals;
displaying to the user device one or more of the available reservations at one or more of the identified hospitals; and
receiving from the user device a selection of one of the available reservations at one of the identified hospitals to book the selected ER reservation.

2. The ER-reservation method of claim 1, wherein the step of identifying hospitals includes receiving user-device location information from the user device and then accessing a database of hospital location information to identify which hospitals are nearby the user device.

3. The ER-reservation method of claim 1, wherein the treatment-category parameter is adult, child/pediatric, urgent care, direct admit, walk-in, or worker's comp.

4. The ER-reservation method of claim 1, wherein the queue-process step of identifying available reservations includes identifying, at each of the identified hospitals, whether a reservation is available in a current time period based on the capacity characteristics of the identified hospitals for the current time period, and if not then whether a reservation is available in a subsequent time period based on the capacity characteristics of the identified hospital for the subsequent time period, in order to identify an earliest available reservation for each of the identified hospitals.

5. The ER-reservation method of claim 1, wherein at least one of the capacity characteristics is wait time, black-out period, or maximum number of reservations per time period.

6. The ER-reservation method of claim 1, further comprising, upon receiving an update to one of the capacity characteristics from a hospital electronic device, sending a reservation reschedule request to the user device.

7. The ER-reservation method of claim 6, further comprising repeating the queue process based on the updated capacity characteristic.

8. A computer-readable non-transitory memory device storing instructions for carrying out the steps of claim 1.

9. A computer-implemented method for booking an ER reservation with a user electronic device, comprising:
receiving at least one hospital search parameter from the user device;
identifying hospitals that meet the hospital search parameter;
receiving at least one treatment-category parameter from the user device, wherein the treatment-category parameter is adult, child/pediatric, urgent care, direct admit, walk-in, or worker's comp;
performing a queue process that includes identifying, at each of the identified hospitals, an earliest available reservation that meets the treatment-category parameter based on one or more capacity characteristics of the identified hospitals by identifying, at each of the identified hospitals, whether a reservation is available in a current time period based on the capacity characteristics of the identified hospitals for the current time period, and if not then whether a reservation is available in a subsequent time period based on the capacity characteristics of the identified hospital for the subsequent time period, wherein at least one of the capacity characteristics is wait time, black-out period, or maximum number of reservations per time period;
displaying to the user device one or more of the earliest available reservations at one or more of the identified hospitals;
receiving from the user device a selection of one of the earliest available reservations at one of the identified hospitals to book the selected ER reservation;
upon receiving an update to one of the capacity characteristics from a hospital electronic device, sending a reservation reschedule request to the user device; and
repeating the queue process based on the updated capacity characteristic.

10. A computer-readable non-transitory memory device storing instructions for carrying out the steps of claim 9.

11. A computer system for booking an ER reservation, comprising:
a server that includes a memory device and that communicates with a user electronic device and a hospital electronic device, wherein the memory device stores instructions for:
identifying hospitals that meet at least one hospital search parameter received from the user device;
performing a queue process that includes identifying, at each of the identified hospitals, an earliest available reservation that meets at least one treatment-category parameter received from the user device based on one or more capacity characteristics received from the identified hospitals;
booking the ER reservation by receiving from the user device a selection of one of the earliest available reservations at one of the identified hospitals displayed to the user device.

12. The ER-reservation system of claim 11, wherein identifying hospitals includes receiving user-device location information from the user device and then accessing a database of hospital location information to identify which hospitals are nearby the user device.

13. The ER-reservation system of claim 11, wherein identifying earliest available reservations includes identifying, at each of the identified hospitals, whether a reservation is available in a current time period based on the capacity characteristics of the identified hospitals for the current time period, and if not then whether a reservation is available in a subsequent time period based on the capacity characteristics of the identified hospital for the subsequent time period.

14. The ER-reservation system of claim 11, wherein the memory device stores further instructions for ranking the earliest available reservations at the identified hospitals to determine a recommended one of the earliest available reservations that is earliest and can be met, then displaying to the user device the recommended one of the earliest available reservations.

15. The ER-reservation system of claim 11, wherein the treatment category parameter is adult, child/pediatric, urgent care, direct admit, walk-in, or worker's comp.

16. The ER-reservation system of claim 11, wherein at least one of the capacity characteristics is wait time, black-out period, or maximum number of reservations per time period.

17. The ER-reservation system of claim 11, wherein the memory device stores further instructions for displaying to the hospital device controls for updating the capacity characteristics of each of the treatment categories.

18. The ER-reservation system of claim 17, wherein the memory device stores further instructions for, upon receiving an update to one of the capacity characteristics from the hospital electronic device, sending a reservation reschedule request to the user device.

19. The ER-reservation system of claim 18, wherein the memory device stores further instructions for repeating the queue process based on the updated capacity characteristic.

20. The ER-reservation system of claim 11, wherein the memory device stores further instructions for displaying to the hospital device controls for updating an open/close status of each of the treatment categories, and for, upon receiving an update to the status of one of the capacity characteristics from the hospital electronic device, sending a reservation reschedule request to the user device.
